(19) United States
(12) Patent Application Publication
(10) Pub. No.: US 2004/0036837 A1
(43) Pub. Date: Feb. 26, 2004

(54) Method and apparatus for measuring aberratio of human eye

(76) Inventor: Jicang He, Beijing (CN)

Correspondence Address: LADAS & PARRY, 26 WEST 61ST STREET, NEW YORK, NY, 10023, US

(21) Appl. No.: 10/362,391

(22) PCT Filed: Aug. 22, 2001
(86) PCT No.: PCT/CN01/01266

(30) Foreign Application Priority Data
Aug. 22, 2000 (CN) 0012357.0

(51) Int. Cl. A61B 3/00

(52) U.S. Cl. 351/200

(57) ABSTRACT

The present invention provides a method and an apparatus for measuring aberration of human eyes. The apparatus comprises three optical paths used respectively for tracing light, measuring light tracer and monitoring pupil. The entrance pupil position of the tracer light is controlled by the array of light source which is conjugated with the pupil plane, and the light recording is comprised by a test-object on a display screen which is conjugated with the fundus oculi. According to the theory used in the invention that to separate the optical paths respectively for tracing light and recording light tracer, the optical paths in the measuring apparatus is shorten, and the brightness of the light source is increased, thereby to avoid the drawback in the prior art that the brightness of the light source is not enough and to ensure has good measurement accuracy. The apparatus has more simple structure, better stability, more easy to be adjusted, and has economic cost.

The present invention relates to a measuring method and a measuring apparatus used in medicine, and particularly to a measuring method and a measuring apparatus for measuring the aberration of human eyes.

Human eyes constituting an optical system can form an image of an external object on the retinas of the fundus oculi. For an ideal eye, the image of an object can be formed quite sharply on the retinas and the contrast of the light distribution of the object surface on the retinas can be kept unchanged. At the same time, the light beams are emitted (or reflected) from a point on the surface of the object, pass through the pupil of the human eye and are focused on a corresponding point on the retina. But the ordinary eyes of human beings are not so ideal as stated above, the light beams passing through the pupil are not in focus, instead, they are out of focus and form a facular light spot, a part of which reach the ideal point but most of which deviates from it, this kind of phenomenon of light beam deviation due to deficiency of the optical system of the eye is called as aberration. As the deficiency of the human eye system, the human eye aberration has serious adverse effect on the human eyesight, especially it has even more adverse effect on the eyesight of the myopic patients.

To compensate the aberration of the human eyes, at first the aberration of the human eyes should be precisely measured. Nowadays, there are two kinds of methods for measuring the aberration of the human eyes, i.e. subjective measurement and objective measurement. As the subjective measuring method has higher precision, good stability, no requirement of scattering the pupil and low cost, so it has become a popular measuring method. Chinese patent No. 98125083.1 provides an apparatus and a method for measuring the aberration of the human eyes, wherein the apparatus and method are capable of measuring the aberration of the human eyes. But the measuring optical path of that invention should perform both the tasks of tracking the light beams and measuring the light traces. In the case of ensuring the precision of measurements, in order to perform both these two tasks, the distance of the whole optical path should be very long, which results in that the brightness of the light emitting diodes is seriously insufficient. In most cases the persons to be tested are still unable to see clearly the target so that the measurement cannot be performed even the super-brilliant light emitting diodes are used.

The object of the present invention is to provide a method and an apparatus for measuring the aberration of the human eyes, the method and apparatus for measuring the aberration of the human eyes separate the task of tracking the light beams and measuring the light traces into two, each of them is executed by one individual optical path, thus de-emphasizing the requirements on the brightness of the light emitting diodes and reducing the loss of the optical energy in the process of optical path transmission, thus resulting in solving the problem of insufficient brightness of the light emitting diodes without affecting the precision of the measurements.

The object of the present invention is achieved by providing a subjective method for measuring the aberration of the human eyes, the method comprising the following steps:

a. A unit light source is randomly chosen from an array light source to be used to emit light beams, the emitted light beams pass through a fixed light-transmitting visual mark and the corresponding optical elements, and form an image of the fixed light-transmitting visual mark on the fundus oculi of a human eye, thus performing light beam tracking;

b. The visual mark on the display screen forms an image on the human eye fundus oculi with the help of the corresponding optical elements, and the light trace registration is performed by moving the visual mark on the display screen, letting it coincide with the fixed visual mark and registration the position of the visual mark on the display screen;

c. Another unit light source is randomly chosen from the array of light source to be used to emit light beams, the emitted light beams pass through a fixed light-transmitting visual mark and the corresponding optical elements, and form an image of the fixed light-transmitting visual mark on the fundus oculi of a human eye, thus performing light beam tracking;

d. The light trace registration is performed by moving the visual mark on the display screen, letting it coincide with the fixed visual mark and registration the position of the visual mark on the screen;

e. Different position data of the visual mark of the display screen are analyzed and the aberration data are obtained by using a numerical calculating method.

Step c and step d can be repeated before the execution of step e to obtain a plurality of position data of visual mark on the display screen, and then step e is executed.

The numerical calculating method used in step e can be an analytical method of least square deviation.

When step c and step d are repeated, each unit light source of the array light source can be lit up randomly one by one.

Before step a is executed, an infra-red light beam can be used to irradiate the pupil of a human eye, with the help of the corresponding optical elements the pupil of the human eye and the concentric circle differentiating plate of the monitoring optical path have their images formed on the electronic video camera simultaneously, which are displayed on the second display screen of the computer control device and have the same optical axis.

Said array light source can be an array of light emitting diodes, or an array of optical fibers or a liquid crystal display panel.

Furthermore, the present invention provides a subjective human eye aberration measuring device, which has the optical paths for tracking the light beams and for registration the light traces, the light source of the optical path for light-tracking is set as an array light source which is conjugate with the pupil plane and connected to the computer control device, in the optical path there is a fixed light-transmitting visual mark which is conjugate with the fundus oculi and forms an image on the fundus oculi of the human eye by the irradiation of any one of the unit light sources in the array of the light sources and the operation of the corresponding optical elements; the light source of the optical path for light trace registration is set as a display screen which is conjugate with the fundus oculi, the visual mark of the display screen forms an image on the fundus oculi and is connected to the movement control device driver on the computer control device; the path for light-tracking and the path for light trace registration coincide with each other before they reach the human eye. The subjective human eye aberration measuring device also comprises a pupil monitoring optical path, which has an infra-red light source set adjacent to the human eye and a concentric circle differentiating plate connected to the electronic video camera of the computer control device, the concentric circle differentiating plate and the photosensitive plane of the electronic video camera are conjugate with the pupil plane, the pupil of the human eye and the concentric circle differentiating plate form images on the second display screen of the computer control device simultaneously with the help of the corresponding optical elements. Said array light source may be a two-dimensional array composed of 10 through 100 unit light sources. Said array light source may be a two-dimensional array composed of 13, 21 or 37 unit light sources. Said array light source can be an array of light emitting diodes, or an array of optical fibers or a liquid crystal display panel. The optical aperture on the pupil plane of each of said unit light sources is not larger than 1 mm. Said computer control device may be a computer, a single-chip machine or an industrial control machine. Said display screen may be a liquid crystal display panel or a small-size display screen. Said movement control device may be a mouse, a joystick or a notebook-type mouse. Said electronic video camera may be an electronic eye, a digital camera or a video camera. Said optical path for light trace registration has a visual chart cross or an image cross formed in conjugation with the fundus oculi before it coincides with the optical path for light-tracking.

The principle of the present invention is as follows: the subjective human eye aberration measuring system comprises an optical path for light-tracking and an optical path for light trace registration, the optical path for light-tracking starting from the array light source, forming images by the optical system and focusing on the pupil plane of the human eye through the light-transmitting visual mark which is conjugate with the fundus oculi. The computer control device selects a unit light source by using the light source driving circuit. At this time, the light beams only enter the human eye through one particular point on the pupil plane. Because the fixed light-transmitting visual mark is conjugate with the fundus oculi, the image of the light-transmitting visual mark seen by the person is actually composed of all the light beams passing through the particular point on the pupil plane. If at this time the computer control device controls another unit light source to emit light, then the image of the light-transmitting visual mark on the fundus oculi is composed of the light beams passing through another particular point on the pupil plane. If the human eye can focus correctly, then there will be no aberration and the images of the visual markes, i.e. The light traces of the light beams entering the human eye through different positions of pupil, will remain unchanged. But due to the aberration of the human eyes, the image of the visual mark will move to another position on the fundus oculi, i.e. The light traces will change. Moreover, the amount of change of the light traces is directly proportional to the aberration at the position of the pupil. At this time, the effect of the aberration can be seen by the person being tested, but cannot be quantitatively determined. The optical path for light trace registration performs the task of determining the aberration. In the optical path for light trace registration, a display screen forms an image at the fundus oculi of the person being tested so that the person being tested can see the light-transmitting visual mark in the optical path for light-tracking and at the same time the visual mark of the display screen in the optical path for light trace registration. Afterwards, the person being tested can operate the movement control device to let the visual mark of the display screen and the visual mark of the fixed transmitting visual mark coincide with each other and use the movement control device to record the position of the light-transmitting visual mark under the control of a program, thus performing the task of registration the light traces. Thereby, the displacement of the light-transmitting visual mark caused by the aberration is transformed into a displacement of the visual mark on the display screen, which can be thus determined. Then, a change of the visual angle can be determined from the measured parameters and a two-dimensional wave aberration distribution and the components of the multiple discrete aberrations can be determined by using a numerical calculating method.

The measuring method for measuring the aberration of the human eyes and the measuring apparatus thereof of the present invention have the following advantages:

1. When the measuring method for measuring the aberration of the human eyes and the measuring apparatus thereof of the present invention are used, the optical path for light-tracking and the optical path for light trace registration are separated, the optical path for light trace registration controls the precision of the measurement and the optical path for light-tracking controls the brightness, thus ensuring enough brightness without affecting the precision of measurement.

2. Moreover, the principle of separating the optical paths for tracking the light beams and for registration the light traces makes the structure of the measuring apparatus simpler, its adjustment more convenient, the stability better and cost lower.

3. The use of the electronic video camera and the concentric circle differentiating plate in the pupil monitoring optical path simplifies the structure of the optical paths and the simultaneous display of the pupil and the concentric circle differentiating plate on the display screen is beneficial for the co-axial control of the positions of the pupil and the optical paths of the apparatus.

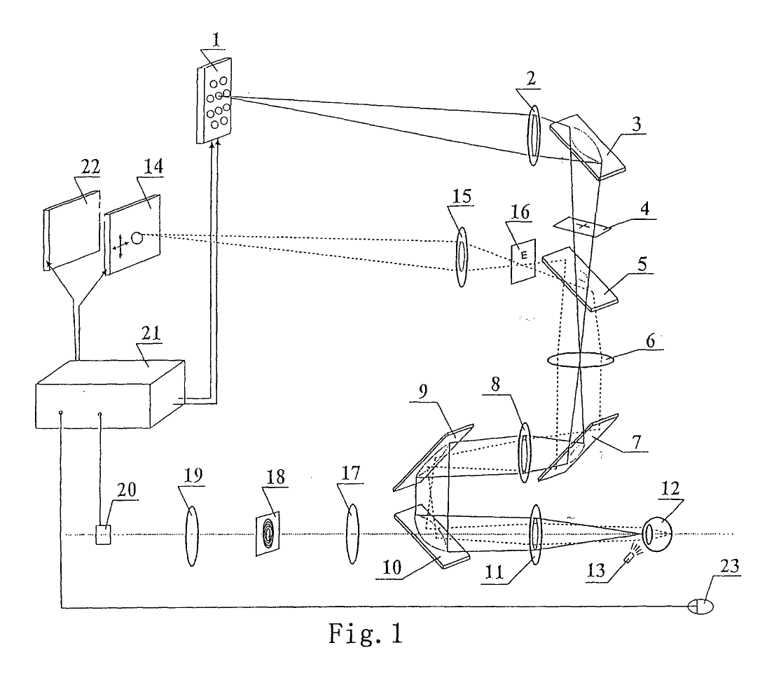

FIG. 1 is the overall schematic configuration diagram of an embodiment of the human eye aberration measuring apparatus of the present invention.

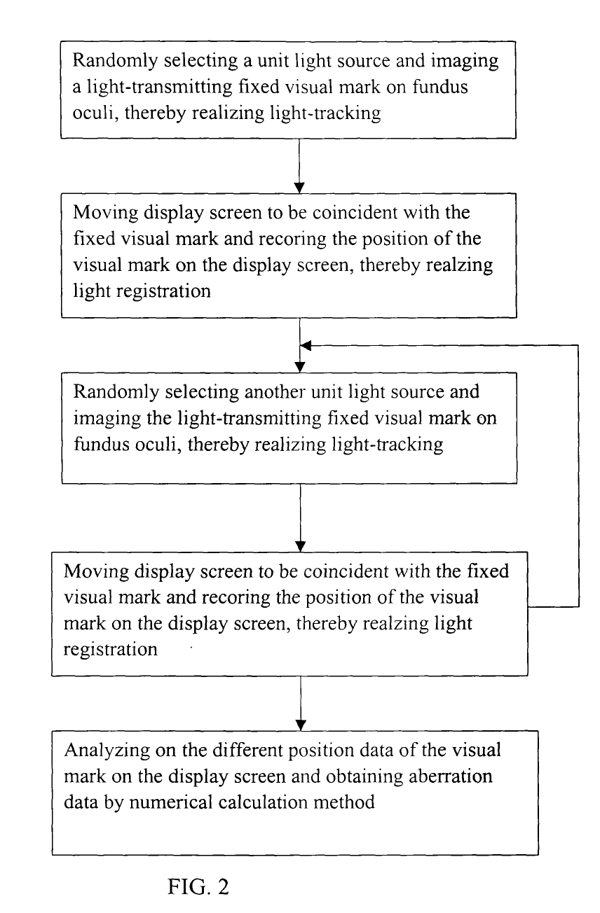

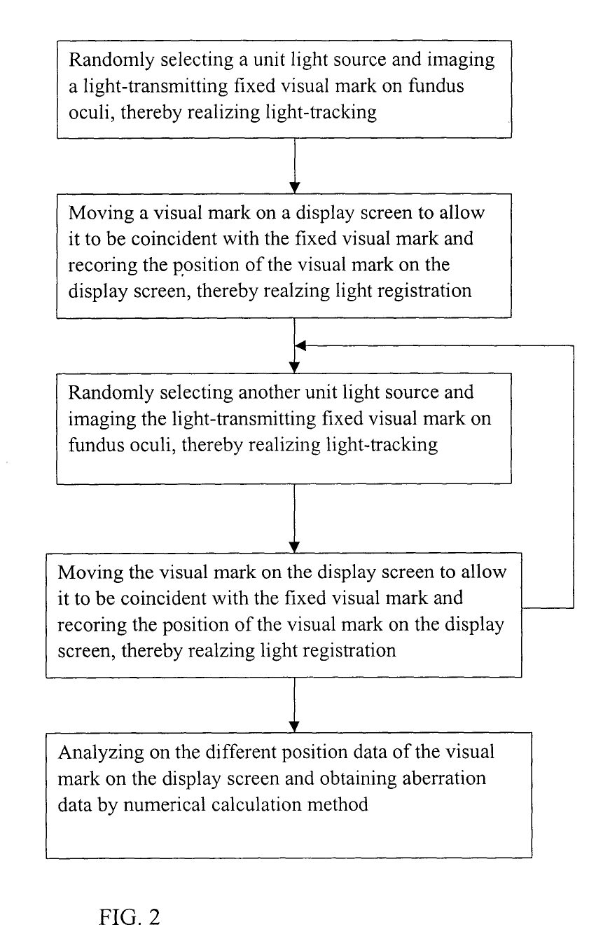

FIG. 2 is a flow diagram of the subjective human eye aberration measuring method of the present invention.

Below there will be more detailed description of the present invention with reference to the accompanying figures:

As shown in FIG. 1, the subjective human eye aberration measuring apparatus of an embodiment of the present invention comprises an array light source 1, a first lens 2, a first reflecting mirror 3, a light-transmitting visual mark 4, a beam splitter 5, a second lens 6, a second reflecting mirror 7, a third lens 8, the third reflecting mirror 9, a cold light reflecting mirror 10, a fourth lens 11, a first computer display screen 14, a fifth lens 15, a visual chart mark 16, a sixth lens 17, a concentric circle differentiating plate 18, a seventh lens 19, an electronic eye CCD 20, a computer and light emitting diode driving circuit 13. The subjective human eye aberration measuring apparatus of an embodiment of the present invention comprises an optical path for light-tracking and an optical path for light trace registration. In an embodiment, the light source of the optical path for light-tracking is set as an array of the light emitting diodes which is conjugate with the pupil plane and connected to the computer. The fixed light-transmitting visual mark 4, which exists in this optical path and is conjugate with the fundus oculi, forms an image at the fundus oculi of the human eye under the irradiation of one of the light emitting diodes of the array light source 1 with the help of the corresponding optical elements.

The concrete configuration is as follows: the optical path for light-tracking has sequentially an array of the light sources 1, the first lens 2, the first reflector 3, a light-transmitting visual mark 4, an beam splitter 5, the second lens 6, the second reflector 7, the third lens 8, the third reflector 9, a cold light reflector 10 and the fourth lens 11, then the light beams enter the human eye 12.

In the embodiment of the present invention, the optical path for light trace registration comprises a computer display screen 14, which is conjugate with the fundus oculi, forms an image of the computer cross of the display screen on the fundus oculi of the human eye with the help of the corresponding optical elements and is connected to the computer 21, and performs the driving operation with the help of the movement control device of the computer. For example, in an embodiment, the visual mark is a cursor of a mouse, but it can be adjusted to be a cross. The optical path for light trace registration has the first computer display screen 14, the fifth lens 15, the visual chart mark 16, the beam splitter 5, the second lens 6, the second reflector 7, the third lens 8, the third reflector 9, the cold light reflector 10 and the fourth lens 11, arranged in this order. The optical path for light-tracking and the optical path for light trace registration partly coincide with each other.

The subjective human eye aberration measuring device also has an optical path for monitoring pupil. The optical path for monitoring pupil has an infra-red light emitting diodes 13 arranged near the human eye and used as an infra-red light source. In the optical path there are also a concentric circle differentiating plate 18 and a CCD electronic eye 20 connected to the computer. The optical path is configured to include: the infra-red light source 13, the fourth lens 11 and a cold light reflector 10 (as a part in which with the optical path for light-tracking and the optical path for light trace registration are coincident with each other), the sixth lens 17, the concentric circle differentiating plate 18, the seventh lens 19 and the electronic eye CCD 20. The concentric circle differentiating plate 18 and the photosensitive surface of the CCD electronic eye 20 are conjugate with the pupil plane, and the human eye pupil and the concentric circle differentiating plate form images on the second display screen 22 of the computer by corresponding optical elements. Through a moving device (not shown), the optical axis of the device and the optical axis of the human eye are aligned based on the display on the second display screen 22.

Said array light source 1 may be a two-dimensional array composed of, for example, 13, 21 or 37 LEDs (light-emitting diodes). The optical aperture of one of said light-emitting diodes on the pupil plane is not larger than 1 mm. As a computer control device, said computer may be replaced with a single-chip machine or an industrial control machine. Said display screen may be a liquid crystal display panel, CRT, etc. As a movement control device, said mouse may be replaced with a joystick or a remote controller or the like. As an electronic video camera, said CCD electronic eye may be replaced with a digital camera or a video camera. As an array light source, said array of LEDs may be replaced with an array of optical fibers (on the array of the optical fibers there is an array of unit light sources) or a point light source two-dimensionally driven by a servomotor.

In the subjective human eye aberration measuring device of the present invention, the optical path for light-tracking starts from the array light source 1, and an image is formed by the optical system and is focused on the eye pupil plane after the light beams pass through the light-transmitting visual mark 4 which is conjugate with the fundus oculi. The computer, using the light source driving circuit, selectively lights up one of the light emitting diodes in the array light source 1, and the light beams only enter the human eye through one particular point on the pupil plane. As the light-transmitting visual cross 4 is conjugate with the fundus oculi, the image of the visual cross seen by the human eye is composed of all the light beams passing through the particular point on the pupil plane. If this time the computer controls another of the light emitting diodes to emit light, the image of the visual cross on the fundus oculi is composed of all the light beams entering the human eye through another particular point of the pupil. If the human eye focuses correctly, then there is no aberration, the image of the visual cross will remain unmoved, i.e. the image formed by the light rays, which enter the human eye from different directions at the pupil, on the fundus oculi will remain unchanged too. But due to the effect of the human eye aberration, the image of the visual cross will move to another position on the fundus oculi, i.e. The position of the image will change. Moreover, the amount of change of the position will increase as the aberration of the eye increases. At this time, the effect of the aberration appears as a change of the position of the image of the visual cross seen by the person being tested, and the change is quantitatively measured by the optical path for light trace registration. In the optical path for light trace registration, a visual cross on the computer display screen 14 (such as a cursor shaped as a cross) forms an image on the fundus oculi of the person being tested. In such a way the person being tested can see the light-transmitting visual mark 4 in the optical path for tracking the light traces and at the same time see the visual mark on the computer display screen in the optical path for light trace registration. Then, the person being tested can move the visual mark on the computer display screen to make the visual cross to be in coincident with the light-transmitting visual mark by moving for example a mouse 23, and can record, with the computer visual cross, the position of the image of the light-transmitting visual mark formed on the fundus oculi by clicking the mouse, thus realizing the registration of the light traces. Thus, the displacement of the image of the light-transmitting visual mark caused by the aberration is transformed into a displacement of the visual mark on the computer display screen and is measured. Then the change of the visual angle is obtained from the results of the measurement, and the two-dimensional wave aberration distribution and the multiple discrete aberration components are calculated by using numerical calculating method.

The human eye pupil 12 forms an image on the post-focusing plane of the lens 11 under the irradiation of the infrared light emitting diode 13 through the lens 11 and the cold reflector 10, the image being coincident with the concentric circle differentiating plate 18. The concentric circle differentiating plate 18 and the image of the human eye pupil form images on the electronic eye CCD through the lens 19, and are displayed on the second display screen 22 of the computer simultaneously for controlling the aligning adjustment of the optical axes of the human eye and the measuring device.

The cold light reflector 10 has the characteristics of reflecting the visual light and transmitting the infrared light.

The human eye aberration measuring device of the present invention separates the dual task of tracking the light beams and measuring the light traces into two tasks to be performed by two optical paths, thus de-emphasizing the requirements on the brightness of the light emitting diodes in the process of the measurement and at the same time reducing the loss of the optical energy in the process of optical path transmission, resulting in solving the problem of insufficient brightness of the light emitting diodes without affecting the precision of the measurements.

In FIG. 2 is shown a flow diagram of the subjective human eye aberration measuring method.

The subjective human eye aberration measuring method of the present invention comprises the following steps:

a. Choosing a unit light-emitting element of the light source is chosen from an array light source to be used to emit light, the emitted light beams pass through a fixed light-transmitting visual mark and the corresponding optical elements, and form an image of the fixed light-transmitting visual mark on the fundus oculi of a human eye, thus performing light beam tracking;

B. The visual mark on the display screen forms an image on the fundus oculi with the help of the corresponding optical elements, and the light trace registration is performed by the person being tested operating display screen visual mark moving device such as a mouse to move the visual mark on the display screen to let it coincide with the fixed visual mark, and by his inputting operation (such as clicking a mouse) to record the position of the visual mark on the screen;

C. Another unit is chosen from the array light source to be used to emit light, the emitted light beams pass through a fixed light-transmitting visual mark and the corresponding optical elements, and form an image of the fixed light-transmitting visual mark on the fundus oculi of a human eye, thus performing light beam tracking;

D. The person being tested performs the light trace registration by using the display screen visual mark movement control device (such as moving the mouse) to move the visual mark on the display screen until it coincides with the fixed visual mark and by inputting operation (such as clicking a mouse) to record the position of the visual mark on the screen;

E. Different position data of the visual mark on the display screen are analyzed and the aberration data are obtained by using a numerical calculating method.

Step c and step d can be repeated before step e is executed to obtain a plurality of position data of visual mark on the display screen, and then step e is executed. The numerical calculating method used in step e may be an analytical method of least square deviation.

When step c through step d are repeated, different unit light sources of the array light source can be lit up randomly.

Before step a is executed, an infra-red light source can be used to irradiate the pupil of a human eye, with the help of the corresponding optical elements the pupil of the human eye and the concentric circle differentiating plate have their images formed on the electronic video camera simultaneously, which are displayed on the second display screen of the computer control device and have the same optical axis.

The measuring method of the present invention is most preferably repeating step c through step d before step e is executed to obtain a plurality of position data of visual mark on the display screen, and then step e is executed. When step c through step d are repeated, each of light emitting diodes of the array of the light sources can be lit up in random order one by one.

There are many kinds of the numerical calculating methods available in step e, it may be an analytical method of least square deviation or other methods.

The selection of the unit light source in step a and step c may be random, arbitrary, or in pre-determined order.

The array of the light sources may be replaced with other suitable configuration of light sources, for example the point light source (such as a laser) driven by a servomotor and moving in a plane perpendicular to the optical axis (i.e. corresponding to the plane in which the array of the light sources exists), i.e. the point light source movable in the plane in which a light source exists.

1. A subjective human eye aberration measuring method, comprising the following steps:
a. A unit light source is randomly chosen from an array light source to be used to emit light beams, the emitted light beams pass through a fixed light-transmitting visual mark and the corresponding optical elements, and form an image of the fixed light-transmitting visual mark on the fundus oculi of a human eye, thus performing light beam tracking;
b. The visual mark on the display screen forms an image on the fundus oculi with the help of the corresponding optical elements, and the light trace registration is performed by moving the visual mark on the display screen, letting it coincide with the fixed visual mark and registration the position of the visual mark on the display screen;
c. Another unit light source is randomly chosen from the array of light source to be used to emit light beams, the emitted light beams pass through a fixed light-transmitting visual mark and the corresponding optical elements, and form an image of the fixed light-transmitting visual mark on the fundus oculi of a human eye, thus performing light beam tracking;
d. The light trace registration is performed by moving the visual mark on the display screen, letting it coincide with the fixed visual mark and registration the position of the visual mark on the screen;
e. Different position data of the visual mark of the display screen are analyzed and the aberration data are obtained by using a numerical calculating method.

2. A subjective human eye aberration measuring method as set forth by claim 1, characterized in that step c and step d can be repeated before step e is executed to obtain a plurality of position data of visual mark on the display screen, and then step e is executed.

3. A subjective human eye aberration measuring method as set forth by claim 1, characterized in that the numerical calculating method used in step e may be an analytical method of least square deviation.

4. A subjective human eye aberration measuring method as set forth by claim 2, characterized in that when step c through step d are repeated, each unit light source of the array light source can be lit up randomly one by one.

5. A subjective human eye aberration measuring method as set forth by claim 1, characterized in that before step a is executed, an infa-red light beam can be used to irradiate the pupil of a human eye, with the help of the corresponding optical elements the pupil of the human eye and the concentric circle differentiating plate have their images formed on the electronic video camera simultaneously, which are displayed on the second display screen of the computer control device and have the same optical axis.

6. A subjective human eye aberration measuring method as set forth by claim 1, characterized in that said array light source may be an array of light emitting diodes, or an array of optical fibers or a liquid crystal display panel.

7. A subjective human eye aberration measuring apparatus, characterized in that said subjective human eye aberration measuring apparatus has an optical path for tracking light beams and an optical path for registration light traces, the light source of the optical path for tracking light beams is set as an array light source which is conjugate with the pupil plane and connected to the computer control device, in said optical path there is a fixed light-transmitting visual mark which is conjugate with the fundus oculi of the human eye and forms an image on the fundus oculi by the irradiation of the randomly selected unit light source in the array of the light sources and the operation of the corresponding optical elements; the light source of the optical path for light trace registration is set as a display screen which is conjugate with the fundus oculi, the visual mark of the display screen forms an image on the human eye fundus oculi and is connected to the movement control device driver on the computer control device; the path for light-tracking and the path for light trace registration coincide with each other before they reach the human eye.

8. A subjective human eye aberration measuring apparatus as set forth by claim 7, characterized in that said subjective human eye aberration measuring apparatus also comprises a pupil monitoring optical path, said monitoring optical path has an infra-red light source set adjacent to the human eye and a concentric circle differentiating plate connected to the electronic video camera of the computer control device, the concentric circle differentiating plate and the photosensitive plane of the electronic video camera are conjugate with the pupil plane, the pupil of the human eye and the concentric circle differentiating plate form images on the second display screen of the computer control device simultaneously with the help of the corresponding optical elements.

9. A subjective human eye aberration measuring apparatus as set forth by claim 7, characterized in that said array light source may be a two-dimensional array composed of 10 through 100 unit light sources.

10. A subjective human eye aberration measuring apparatus as set forth by claim 9, characterized in that said array light source may be a two-dimensional array composed of 13, 21 or 37 unit light sources.

11. A subjective human eye aberration measuring apparatus as set forth by claim 7, characterized in that said array light source may be an array of light emitting diodes, or an array of optical fibers or a liquid crystal display panel.

12. A subjective human eye aberration measuring apparatus as set forth by claim 7, characterized in that the optical aperture on the pupil plane of each of said unit light sources is not larger than 1 mm.

13. A subjective human eye aberration measuring apparatus as set forth by claim 7, characterized in that said computer control device may be a computer, a single-chip machine or an industrial control machine.

14. A subjective human eye aberration measuring apparatus as set forth by claim 7, characterized in that said display screen may be a liquid crystal display panel or a small-size display screen.

15. A subjective human eye aberration measuring apparatus as set forth by claim 7, characterized in that said movement control device may be a mouse, a joystick or a notebook-type mouse.

16. A subjective human eye aberration measuring apparatus as set forth by claim 8, characterized in that said electronic video camera may be an electronic eye, a digital camera or a video camera.

17. A subjective human eye aberration measuring apparatus as set forth by claim 7, characterized in that said optical path for light trace registration has a visual chart cross or an image cross formed in conjugation with the fundus oculi before it coincide with the optical path for light-tracking.
